(19) United States
(12) Patent Application Publication
(10) Pub. No.: US 2006/0264979 A1
(43) Pub. Date: Nov. 23, 2006

(54) Novel enhanced vascular surgical device

(76) Inventor: Rudolph C. Shepard, Coto de Caza, CA (US)

Correspondence Address: GREENBERG TRAURIG LLP, 2450 COLORADO AVENUE, SUITE 400E, SANTA MONICA, CA, 90404, US

(21) Appl. No.: 11/134,665

(22) Filed: May 20, 2005

(51) Int. Cl. A61B 17/08

(52) U.S. Cl. 606/151

(57) ABSTRACT

An improved stabilizing tool functions in complement with conventional vascular surgical, interventional, cardiological and urological medical radiological, devices among others. Provided is a combinational set of tubular housing elements having alternate fail-safe securement mechanisms which prevents contamination while being operable, generally by a skilled surgeon with one of his two hands, leaving the other free to perform required surgical tasks.

### FIELD OF THE DISCLOSURE

The present disclosure generally relates to vascular surgical devices, products and processes for using and making such items.

In particular, the present disclosure relates to the provision of novel enhanced vascular surgical devices which secure, for example, known catheter-based systems during surgery simply without adding cost or time constraints, including fail-safe securement mechanisms with added benefits for safety purposes.

### BACKGROUND OF THE DISCLOSURE

Many vascular surgical and interventional cardiological procedures involve introducers, catheters and related tubular devices which need to be secured during the procedures. Several examples provide context demonstrative of the longstanding need for improved securement devices. It is respectfully submitted that both during open surgery and minimally invasive procedures the present disclosure has functional utility.

For example, any vascular surgical procedure which involves treating or replacing a compromised vessel with a graft, or replacement segment requires a high degree of stability for the instruments and devices being used to emplace the device within the patient. This longstanding need remains largely unaddressed among the prior art, while being squarely addressed by the teachings of the present disclosure.

Vascular surgical procedures often involve extremely precise incisions and a high degree of risk of blood loss, contamination of subject instruments and implants and plethoric related challenges. The present disclosure targets these shortcomings of the prior art devices and procedures which have become known to the present inventor.

Further, with the advent of minimally invasive procedures, and endovascular techniques it is likely that later developed procedures and devices will likewise be appropriate for use with the teachings of the instant disclosure.

By way of further example, drug-eluting stents are now conventional, yet delivery devices are constantly evolving, and the need for emplacement of grafts, stents, stented and unstented systems, and many other devices are pressing.

Vascular grafts have been used for decades now to bridge challenged vessels. Whether used to enhance the integrity of major vessels, such as the aorta, carotid or in the peripheral vasculature for branched off-shoots of the same (femoral, popliteal, renal arteries and others) the need is generally to have a graft-system that can be swapped-in for a compromised vessel without creating further trauma.

Materials for vascular grafts range from fabrics like dacrons and polytetraflouroethylenes (particularly the expanded version—ePTFE) to natural vessels and various new materials are being tested and developed constantly. In order to situate them complex, often catheter based delivery systems need to be involved. Stability of any type of such a system during surgery is a major issue. The unmet clinical need is addressed by use of the present disclosure during vascular surgery, and any of the other procedures set forth herein, and defined by the claims appended hereto.

The term “stent” is generally used to describe endoprosthetic devices which are implanted in blood vessels or other anatomical passageways of the body for the purpose of treating stenoses, aneurysms, occlusions, etc. Typically, such stents are implanted in blood vessels to maintain dilation and patency of an occluded region of blood vessel, or to bridge a weakened or aneurysmic region of blood vessel. They may be used in conjunction with, in place of, or following other procedures.

On the other hand, some typical peripheral applications of such stents are for the treatment of constrictions or injuries to the gastrointestinal tract (e.g., esophagus), ducts of the biliary tree (e.g., common bile duct) or anatomical passageways of the genitourinary tract (e.g., ureter, urethra, fallopian tube, etc.). Most stents are initially disposed in a compact configuration of relatively small diameter, whereby the stent may be mounted upon or within a delivery catheter for insertion and transluminal advancement into the desired anatomical passageway. There are various configurations commonly known, with tubularity being a common denominator, along with catheter based delivery.

Such stents are radially expandable to a larger “operative” diameter which is equal to or slightly larger than the diameter of the blood vessel or other anatomical passageway in which the stent is to be implanted. When radially expanded to such operative diameter, the stent will typically become released from the delivery catheter and embedded or engaged to the surrounding wall of the blood vessel or other anatomical passageway. This is different from, and to a certain extent the opposite of using a device to bridge an aneurysm, as discussed below.

In the cardiovascular space, some stents are covered with tubular sleeves, in which case they are typically referred to as a “stented graft”. In general, stents and stented grafts fall into two major categories—a) self-expanding and b) pressure-expandable.

Those of the self-expanding variety may be formed of resilient or shape memory material (e.g., spring steel or nitinol®) which is capable of self-expanding from its first (radially compact) diameter to its second (operative) diameter without the exertion of outwardly-directed force against the stent or stented graft. Likewise, specifically characterized by being generally tubular, and requiring trocars, dilators, introducers, and complex catheter systems, stents need precise and stable techniques to ensure proper placement and long-term results in patients.

Catheters for the introduction or removal of fluids may be located in various venous locations and cavities throughout the body of a patient for introduction of fluids to the body or removal of fluids from the body. Such catheterization may be performed by using a single catheter having multiple lumens. A typical multiple lumen catheter is a dual lumen catheter in which one lumen introduces fluid and the other lumen removes fluid. An example of such a multiple lumen catheter assembly are the SPLIT-CATH® brand of catheter, the Fogarty® brand of catheter (Edwards Lifesciences LLC, Irvine, Calif.) and numerous other devices known to artisans. Catheterization may also be performed by using separate, single lumen catheters inserted through two different incisions into an area to be catheterized.

Another type of such a catheter assembly is the TESIO® brand of catheter. Generally, to insert any catheter into a blood vessel, the vessel is identified by aspiration with a long hollow needle in accordance with the well known Seldinger technique. When blood enters a syringe attached to the needle, indicating that the vessel has been found, a thin guide wire is then introduced, typically through a syringe needle or other introducer device into the interior of the vessel. This is in contrast to the endovascular and open surgical procedures which have been described so far, however.

Securement devices are needed by surgeons performing those procedures. The introducer device is then removed, leaving the end portion of the guide wire that has been inserted into the vessel within the vessel and the opposing end of the guide wire projecting beyond the surface of the skin of the patient. At this point, several options are available to a physician for catheter placement. The simplest is to pass a catheter into the vessel directly over the guide wire. The guide wire is then removed, leaving the catheter in position within the vessel. However, this technique is only possible in cases where the catheter is of a relatively small diameter, made of a stiff material, and not significantly larger than the guide wire, for example, for insertion of small diameter dual lumen catheters. If the catheter to be inserted is significantly larger than the guide wire, a dilator and sheath device is passed over the guide wire to enlarge the hole.

The dilator and the guide wire are then removed from the sheath, leaving only the sheath. The catheter is then inserted through the sheath and into the vessel. The sheath is then removed from around the catheter by tearing the sheath as the sheath is being removed from the patient. For chronic catheterization, in which the catheter is intended to remain inside the patient for an extended period of time, such as for weeks or even months, it is typically desired to subcutaneously tunnel the catheter into a patient using various tunneling techniques. The present device can be used at any point during these types of procedures.

The catheter is typically tunneled into the patient prior to inserting the catheter into the patient's vein. Securement mechanisms provide a distinct advantage in this instance. The subcutaneous tunnel provides a somewhat stable anchor to prevent the proximal end of the catheter from moving and possibly becoming dislodged, which could result in patient injury or death. Surgeons and interventional radiologists have expressed an ongoing interest in improvements to known systems, and have supplied an ongoing litany of needs in these areas.

An anchoring cuff typically circumscribes a portion of the catheter assembly that is located within the tunnel, allowing skin tissue to grow in and around the cuff, further stabilizing the catheter. Catheter assemblies typically include a hub that connects a proximal end of each catheter lumen with a distal end of an extension tube. Occasionally, a blood clot may form in a catheter lumen at a junction between the catheter lumen and the hub, cutting off blood flow through the catheter lumen. Such an occasion typically necessitates removal of the entire catheter assembly from the patient, including insertion of a new catheter assembly in place of the failed catheter assembly.

Part of this removal includes separating the skin from the cuff in the subcutaneous tunnel, and pulling the catheter assembly through the tunnel, as well as removing the catheter lumens from the patient's blood vessel. The removal of the failed catheter assembly and the insertion of the new catheter assembly causes additional trauma to the patient, which is not desired. The need for the instant disclosure is underscored by this and the further examples. Further details may be found in U.S. Letters Pat. Nos. 4,170,995; 4,397,647; 5,382,239; 5,976,192; 6,013,092; 6,375,675; and 6,379,372; each of which are expressly incorporated herein by reference.

Likewise, longstanding among the needs addressed by the instant disclosure, a holder for securement to the body of a patient for the purpose of properly holding a urinary catheter, nasogastric or intravenous tube in place is taught. Artisans will readily understand how to place it and how it works. The various tubes with which the holder may be used will hereinafter be referred to as “catheter tubes” for convenience. The holder is particularly useful for positioning a urinary catheter tube on a patient, or other similar catheter tube which, if pulled longitudinally or rotationally, can cause the patient discomfort or pain as well as physical trauma and dislodgement of the tube.

Various types of catheter tube holders have been proposed by those skilled in the art. Presently in use are arrangements ranging from the simple tape wrapped around a patient's limb over the catheter tube to hold it in place, to more sophisticated holders. The rigged tape arrangements have obvious drawbacks in that they are irritating to a patient, time consuming to apply and remove, and do not always hold the catheter tube properly in place. The adhesive portion of the tape may also cause physical-chemical degeneration of the catheter tube, among the plethoric challenges with such systems.

The tapes further may be contaminated with excrement, blood or other body fluid and cannot be easily cleaned. The various other catheter tube holders proposed are generally rather specialized in function and may not always be generally useful in holding a variety of different catheter tubes in place. This holder is of the general known type of catheter holder which includes a pliant adhesive-bearing base which is much like the flat head of a rubber suction dart or suction cup.

The summary point is that current vascular surgical tools are not completely effective either for immobilizing the complex assemblies used to insert other medical devices into the vessels of patients to maintain patentcy and/or by-pass occlusions or to secure catheters or related tubes before, during or after surgery. According to the present disclosure such a device is shown that will apply to the multiplicity of applications only some of which are expressly disclosed but all others similar, related or known to those skilled in the arts.

To these ends, offered for consideration is a unique device that can be used to secure tubular devices, introducers, pre-loaded grafts—including stented grafts, by a skilled surgeon, usually with only one-hand required, among other things.

Combinations of several utilities in distinct types of systems have yet to adequately address industrially efficient construction processes, products thereby or functionally utile improvements of the same, in this area. The instant disclosure satisfies these longstanding needs and others, and for this reason it is respectfully proposed, constitutes progress in science and the useful arts, and offers for consideration subject matter proper for a U.S. Letters Patent. Such relief is earnestly solicited.

### SUMMARY

Briefly stated, the present disclosure works in conjunction with conventional surgical tools used by vascular surgeons, cardiologists, interventional radiologists and urologists, among others. A novel enhanced vascular surgical device, which comprises, in combination, a first semi-circular trough-like element extended in a longitudinal direction from a first and to a second end, a second semi-circular trough-like element extended in a longitudinal direction from a first to a second end, whereby the second semi-circular trough like element is effective for supporting at least one tubular member from the group consisting of a catheter, an introducer and related vascular surgical tools, and contains a plurality of apertures for receiving matching extensions on the first semi-circular trough like element, and wherein the first semi-circular trough-like element secures the tubular member by the peripheral edges of a lumen defined by joining said first and second elements and a supplement securing pin extends through the first semi-circular trough-like element to the second when assembled.

An improved stabilizing tool functions in complement with conventional vascular surgical, interventional, cardiological and urological medical radiological, devices among others. Provided is a combinational set of tubular housing elements having alternate fail-safe securement mechanisms which prevents contamination while being operable, generally by a skilled surgeon with one of his two hands, leaving the other free to perform required surgical tasks.

Likewise disclosed is a holder device for securement to the body of tubular means, comprising, in combination, for accepting the plurality of male extensions, a first longitudinally extended half-cylinder assembly having a plurality of male extensions thereon for matingly engaging a supplementary site in second match half; and each having a grooved passage for accepting a ring-like securing structure; a second longitudinally extended half cylinder assembly having a first and a second end and having a plurality of female apertures.

Further taught according to the teachings of the present disclosure is a process for stabilizing an instrument set during surgery, which comprises, providing a first half of a securing device having a grooved passageway at a desired site upon a patient, emplacing a catheter or the like tubular member in the grooved passageway, closing the securing device by placing a top half over the tubular member, and adding a fail-safe securing mechanism to first half of the device.

### BRIEF DESCRIPTION OF THE DRAWINGS

Referring now to the drawings for the instant disclosure, which it is noted is described in particularity to those skilled in the art in the appended claims, however, further objectives and advantages of the present disclosure, together with methods of making and using the instant disclosure will be better understood by referring to the following detailed description taken in conjunction with the following illustrations, employing common reference designating elements, wherein:

FIG. 1 is a schematic view of a novel enhanced vascular surgical device, according to embodiments of the instant disclosure, in a closed position;

FIG. 2 is a schematic view of a novel enhanced vascular surgical device, according to embodiments of the instant disclosure in an open position;

FIG. 3 is a detailed schematic view of a novel enhanced vascular surgical device, according to embodiments of the instant disclosure, showing an example of mating engagement, according to embodiments of the present invention;

FIG. 4 is a side view of a novel enhanced vascular surgical device, according to embodiments of the instant disclosure;

FIG. 5 is an open view through a novel enhanced vascular surgical device as shown in FIG. 4, according to embodiments of the instant disclosure;

FIG. 6 is an assembled view of a novel enhanced vascular surgical device, according to embodiments of the instant disclosure;

FIG. 7 is another view of a novel enhanced vascular surgical device, according to embodiments of the instant disclosure;

FIG. 8 is a view of a novel enhanced vascular surgical device, of FIG. 7, according to embodiments of the instant disclosure; and

FIG. 9 is an assembled view of a novel enhanced vascular surgical device, according to embodiments of the instant disclosure.

### DETAILED DESCRIPTION

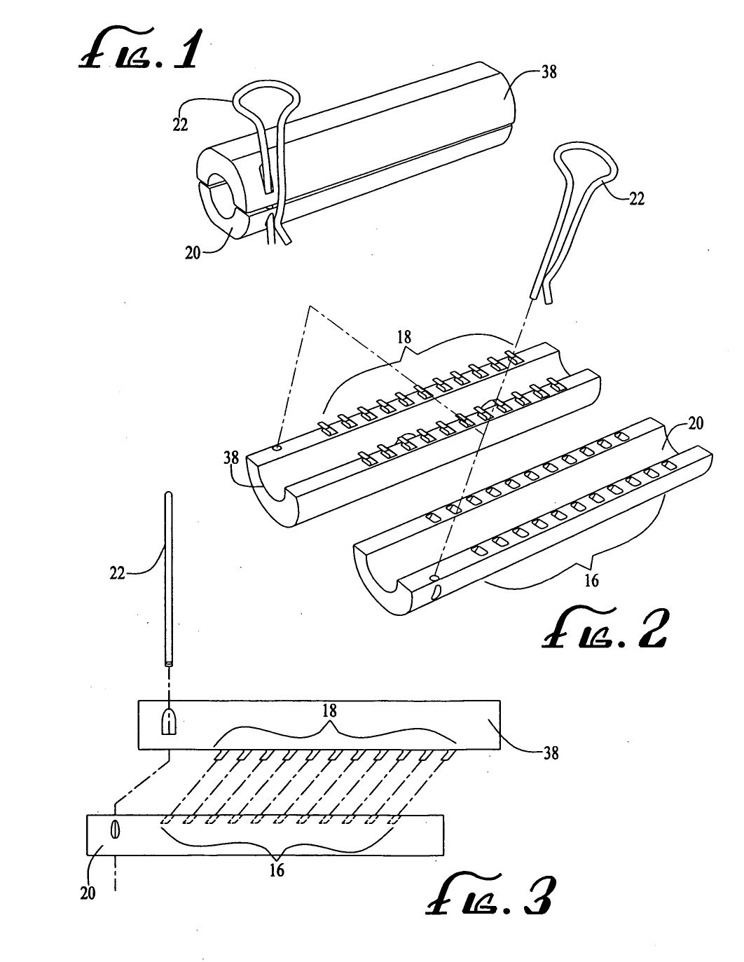

Referring now to FIG. 1, novel enhanced vascular surgical device is characterized generally by first/top element 38 and second/bottom element 20, secured by fail-safe pin 22. In this schematic view the device is shown in a closed, or secured state. This would be used, for example, as described above with any known vascular graft system, or as part of a system styled method and apparatus for positioning an intraluminal graft. More specifically the present disclosure is able to be used for any vascular surgical procedure from vascular graft emplacement balloon angioplasty, or any related treatment.

An artery or other vessel that is weakened by disease, injury, or congenital defect, can become distended due to the pressure of blood or other fluid flowing-through the weakened area. In the vasculature, this distended weakening is called an aneurysm. An aneurysm typically occurs in the arterial vessels of the head, chest, or abdomen. The distension may cause the vessel to rupture, which can have serious, even life-threatening consequences. Once again, surgical correction must be undertaken using delivery systems evolving constantly.

By way of further example, aneurysms in the abdominal aorta are typically distended around the circumference of the aorta and tapered at both ends. Most aneurysms of the abdominal aorta are caused by atherosclerotic weakening of a segment of the wall. Abdominal aneurysms may cause backache and severe pain, and may be visible as a throbbing swelling. If an abdominal aorta ruptures, it is seriously life threatening. Most are not able to be detected and are fatal once they exceed about five centimeters in most people. Newer endovascular procedures have become safe and effective for such issues, but employ many different types of delivery systems, many of which require more stability than currently exists.

Traditionally, aneurysms have been treated by radical surgical graft replacement. This approach is risky for the patient and is sometimes not feasible due to other pre-existing disease states of the patient. More recently, aneurysms have been treated by placement of an intraluminal or endovascular graft. These intraluminal or endovascular grafts may be of various types, including grafts having stents, wireforms, or other attachment means attached to or integrated into the graft structure.

In general, intraluminal grafts and their respective support and/or attachment means fall into two major categories, self-expanding and pressure expandable. Self-expanding intraluminal grafts, are discussed above. Self-expanding material is capable of being formed in a configuration from which it may be compressed to a radially compact diameter for placement within a damaged vessel. At the time of use, the memory feature of these materials causes them to self-expand from the radially compact diameter to the expanded operative diameter.

Pressure-expandable intraluminal grafts are supported and/or attached via plastically deformable material such as stainless steel that is initially formed in its radially compact diameter. This type of material does not have memory, and will remain in the radially compact diameter until manually expanded. Typically, outwardly directed pressure is exerted upon the graft through use of a balloon so as to cause radial expansion and resultant plastic deformation of the material to its operative diameter.

Careful positioning and firm implantation of the intraluminal graft is critical to the successful treatment of the underlying medical condition. This is particularly difficult to do without supplemental securement.

Notwithstanding the important teachings of the prior art and various references, features of the aforementioned procedure have recognized shortcomings that make them less than complete solutions to the treatment of aneurysms in the vasculature, or to the treatment of similar damage to other vessels. The present disclosure provides substantial improvements to the methods and apparatus of the prior art, in terms of provision or supplemental or further catheter/introducer dilatoc/trocar support mechanisms to satisfy longstanding needs.

The present disclosure provides improvements to methods for placing a bifurcated graft at the site of a damaged vessel. In a preferred embodiment, the methods and apparatus of the present disclosure are effective to use during placement of a bifurcated graft within an aneurysm located in the abdominal aorta downstream of the renal arteries. Preferably, placement of the graft is through the right femoral artery of a patient.

FIG. 2 shows an embodiment of the present disclosure with the schematic and exemplary device in an open position. First/top element 38 may be detachably assembled in making engagement with second/bottom element 20 using a plurality of connecting members 18 which become aligned at, and are accepted by matching receiving slots 16.

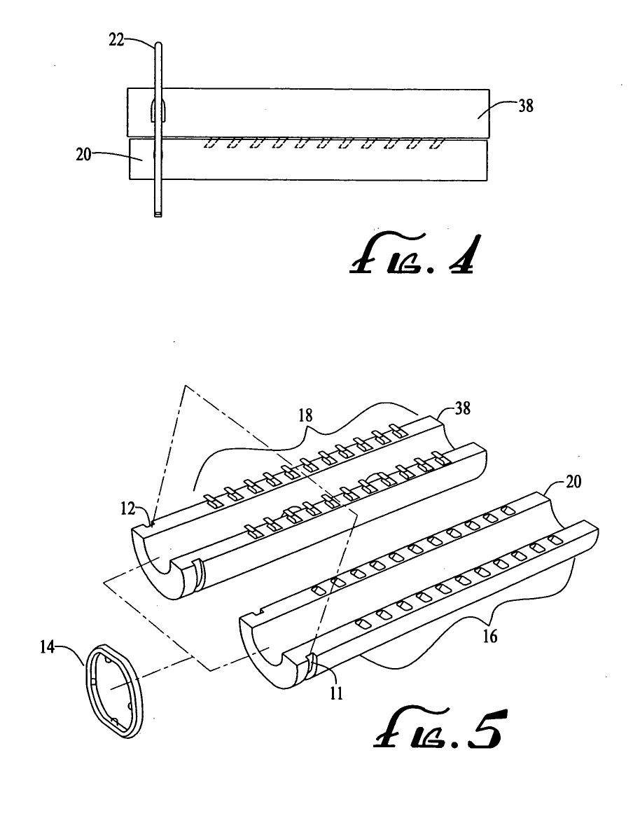

Turning to FIG. 3 and FIG. 4, fail safe pin 22 further secures first/top element 38 to second/bottom element 20, as the device goes from a first position (FIG. 3) to a second position (FIG. 4). FIG. 4 shows second position where first/top element 38 is secured to second/bottom element 20, and fail-safe pin 22 emplaced. Vascular surgeons generally can assemble and secure the device of the present disclosure with one-hand.

Another use is of the endovascular procedure mentioned above. Generally these require an introducer assembly be provided which is configured for placement over a guidewire and for facilitating the advancement of various catheter assemblies required in connection with the practice of the invention. The introducer assembly includes a sheath, valve head, and a dilator. The sheath is preferably cylindrical in shape and is formed so as to have an appropriate flexibility and an outer diameter suitable for placement at the location of an aneurysm to be repaired. The valve head permits insertion and removal of various catheters during the method of the present invention without significant loss of blood from the femoral artery. The proximal end of the valve head is provided with a threaded connector which facilitates connection of the valve head to other catheters. The dilator, which includes a tapered tip, is placed during use through the valve head and the sheath so that the tapered tip portion protrudes from the sheath. The dilator tip portion is capable of being advanced gently through the tortuous pathway of the vasculature without causing undue trauma or a perforation, yet is also sufficiently stiff to cause the blood vessels to assume a less tortuous path.

FIG. 5 shows an alternate securing mechanism, namely elastic ring 14, which is housed by groove 11/12 and may likewise have a series of engagement bumps, steps, or ridges. Typically, one to four lips, bumps, protrusions or the like and the elasticity of securing member 14 provide for a snugly engaged fit. Once again, the surgeon can generally secure the device using only one hand, during any surgical procedure, for example while putting in a graft.

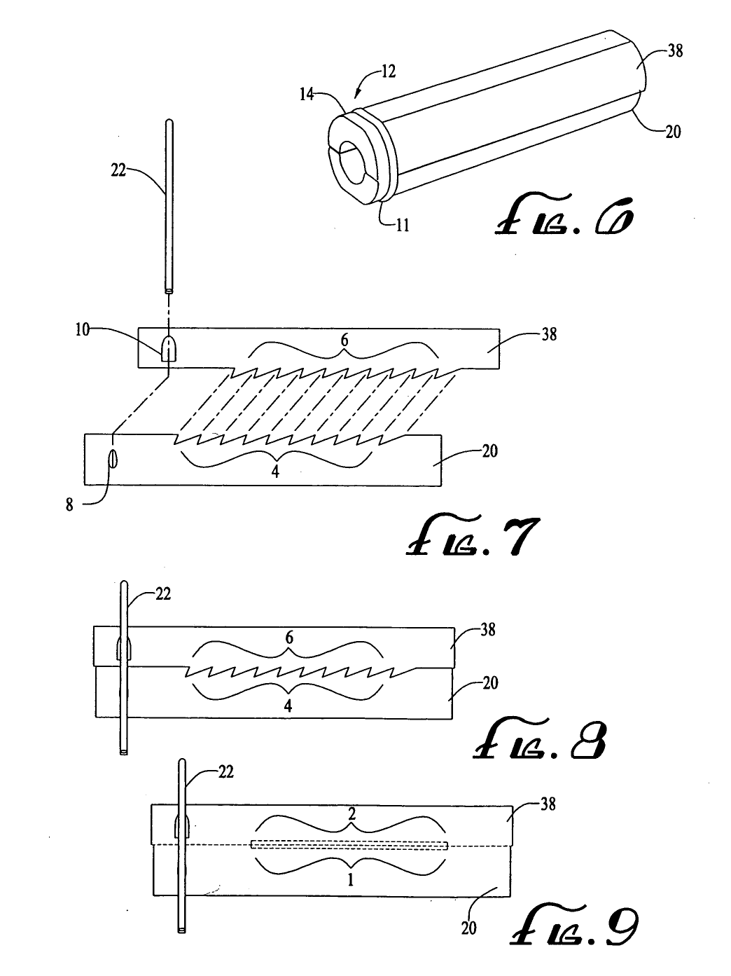

FIG. 6 is another view of the device in a secured state like FIG. 4, with the alternative securing member 14 shown disposed in groove 11/12 with first/top element 38 lodgedly secured to second/bottom element 20. FIG. 7 illustrates, once again the movement from an open to a closed position, whereby the second or closed configuration is used to house the tubular members during surgery. FIG. 8 shows this secured or assembled state with first/top element 38 lodgedly secured to second/bottom element 20, as during surgery, with fail-safe pin 22. Saw tooth engagement zone 4 mates with complementary engagement zone 6 securing elements 38 and 20.

FIG. 9 shows first/top element 38 lodgedly secured to second/bottom element 20 by alternate securement means which may involve nanotechnology, magnetics, or any after developed technology. As in FIG. 8, engagement zone 2, mates with complementary engagement zone to service members 38 and 20.

The present inventor has further discovered that it is possible to use the instant devices for positionally stabilizing a catheter on the body of a patient. Although described herein specifically in terms of a “fully-indwelling” catheter, the present invention encompasses stabilization of intravenous type catheters. First, the device must hold the catheter tube positionally stabilized relative to the device itself. Second, the device itself must be held positionally stabilized relative to the body of the patient. Some devices require conventional adhesive tape to be secured to the patient's skin; such tape, as a general rule, does not hold in place for any considerable period of time and is often time-consuming and bothersome to employ.

Other devices utilize stretch rubber strips with VELCRO® brand of fastening devices wherein the strips encircle the patient's thigh; these are unsatisfactory because, in order to prevent the device from slipping along the thigh, the strips must be wrapped so tightly as to constrict blood circulation. Other devices hold the tube by means of some snap-fit engagement; such devices are subject to displacement of the catheter tube from the device and to movements which inadvertently remove the tube from the snap-fit. Still other devices require that the tube be tied, taped, or similarly engaged to the device; such arrangements tend to restrict the flow through the catheter tube. Finally, there are other devices which require special configurations of catheter tubes to engage the tube properly; such custom tubing requirements are clearly undesirable since it will not be useful for the majority of commercially available catheters.

Fixation devices for securing tubing such as catheter tubing to a patient undergoing medical treatment. At least part of such tubing lies outside of the patient and must be secured to avoid entanglement or crimping which may impede the infusion or monitoring process.

Also featured is use of the instant device for holding tubing such as intravenous catheter tubing or the like to a patient. The device comprises two halves and each of the halves is made up of a channel. The inner surface disposed about the channel is provided with a male/female mating or engagement system. The tubing is gripped between the two channels when the two halves are brought together and the pieces of the engagement system touch each other. The device is provided with a connector which holds the lobes together so that the channels can grip the tubing.

The engagement system may have several embodiments. It may have a contoured gripping surface or it may comprise a tacky material which is designed to grip the tubing. The contoured surface may be sinusoidal, roughened, uneven or it may be made up of a plurality of bumps. The gripping means may also be made up of a liner which likewise may be made of a tacky material or contoured as described above. When the halves are joined, the inside diameter of the tube formed by the channels is typically slightly smaller than the outside diameter of the tubing so that the tubing is gripped by the device without undue constriction.

It should be understood, however, that the present invention contemplates and includes all conventional adjustments in the embodiments described or shown herein (including such adjustments in the size, orientation, proportions, and relative positioning of parts) made to accommodate those differences in designs.

1. A novel enhanced vascular surgical device, which comprises, in combination:
a first semi-circular trough-like element extended in a longitudinal direction from a first and to a second end;
a second semi-circular trough-like element extended in a longitudinal direction from a first to a second end;
whereby the second semi-circular trough like element is effective for supporting at least one tubular member from the group consisting of a catheter, an introducer and related vascular surgical tools, and contains a plurality of apertures for receiving matching extensions on the first semi-circular trough like element;
and wherein the first semi-circular trough-like element secures the tubular member by the peripheral edges of a lumen defined by joining said first and second elements and a supplement securing mechanism further secures the first semi-circular trough-like element to the second when assembled.

2. The novel enhanced vascular surgical device of claim 1, wherein the supplemental securing mechanism comprises a cotter-pin like element extending through an aperture extending through both semi-circular elements when the matching extensions are housed in the plurality of apertures.

3. The novel enhanced vascular surgical device of claim 1, wherein the supplemental securing mechanism comprises a ring-like structure and a plurality of engagement extensions which is aligned in a trough for receiving the same wherein the matching extensions are housed in the plurality of apertures.

4. The device of claim 1, operable by one hand of a user.

5. The device of claim 2, operable by one hand of a user.

6. The device of claim 3, operable by one hand of a user.

7. A stabilizer device for securement to the body of tubular means, comprising, in combination:
a first longitudinally extended half-cylinder assembly having a plurality of male extensions thereon for matingly engaging a supplementary site in second match half;
and each having a grooved passage for accepting a ring-like securing structure;
a second longitudinally extended half cylinder assembly having a first and a second end and having a plurality of female apertures for accepting the plurality of male extensions.

8. The stabilizer device of claim 7, further comprising:
a ring-like securing structure wrappingly engaging the first and second longitudinally extended half-cylinder assemblies by settling in the grooved passageway and maintaining adequate tension to prevent undesired de-coupling of the first and second longitudinally extended half cylinders.

9. The stabilizer device of claim 8, wherein the adequate tension is maintained by stretching the ring-like securing structure such that each of a plurality of raised projection bumps bracingly engages the inner surface located within the grooved passageway.

10. The stabilizer device of claim 9, wherein the material is biocompatible.

11. The stabilizer device of claim 10, wherein the material is a plastic, rubber or composite material.

12. The stabilizer device of claim 11, wherein the material is coated with a lubricious coating.

13. The stabilizer device of claim 12, wherein at least one drug is incorporated with the second longitudinally extended half cylinder assembly which abuttingly engages a surface of a patient's body.

14. A process for stabilizing an instrument set during surgery, which comprises:
providing a first half of a securing device having a grooved passageway at a desired site upon a patient;
emplacing a catheter-like tubular member in the grooved passageway and into a patient;
closing the securing device by placing a second half of a securing device having a grooved passageway over the tubular member; and
adding a fail-safe securing mechanism to secure first half of the device.

15. The process of claim 14, wherein the providing step further comprises the first half of the securing device having female apertures adjacent the grooved passageway to receive male extending members.

16. The process of claim 15, whereby the closing step is followed by an opening step to allow passage of at least one of thrombus, a balloon with the same, an embolic protection device, a basket, a cuff and other mechanisms used to remove clots whereby contamination of the patient is less likely than with known systems.

17. The process of claim 16, whereby the fail-safe securing mechanism is a cotter-pin like member.

18. The process of claim 16, whereby the securing mechanism is a ring-like elastic member.

19. The process of claim 18, wherein the ring like member further comprises a plurality of stepped projections on engagement members.

20. The process of claim 19, further comprising repeating any of the steps.
